(19) United States
(12) Patent Application Publication
(10) Pub. No.: US 2004/0006136 A1
(43) Pub. Date: Jan. 8, 2004

(54) Anthelmintics for preventing parasitic infections in human and animals

(76) Inventors: Achim Harder, Koln (DE); Georg von Samson-Himmelstjerna, Mellendorf (DE); Bernd-Wieland Kruger, Bergisch Gladbach (DE); Heinz Mehlhorn, Neuss (DE); Jurgen Schmidt, Dusseldorf (DE)

Correspondence Address: JEFFREY M. GREENMAN, BAYER PHARMACEUTICALS CORPORATION, 400 MORGAN LANE, WEST HAVEN, CT, 06516, US

(21) Appl. No.: 10/311,486

(22) PCT Filed: Jun. 26, 2001
(86) PCT No.: PCT/EP01/07296

(30) Foreign Application Priority Data
Jul. 6, 2000 (DE) 100 32 880.6

(51) Int. Cl. A61K 31/24; A61K 31/198

(52) U.S. Cl. 514/534; 514/563

(57) ABSTRACT

The invention relates to agents containing specific active ingredients that act as repellents and to their use for preventing humans or animals from being infected with parasitic flatworms (platyhelminths).

The present invention relates to compositions comprising certain active compounds which are suitable as repellents, and to their use for preventing an infection of humans or of animals by the infectious stages of parasitic flatworms (platyhelminths). The compositions are used on the skin against the flatworm stages which are capable of penetrating through the skin into the host organism (cercariae).

Several platyhelminth species cause serious diseases in humans and animals. In tropical countries, infections with Schistosoma species in particular cause chronic suffering and frequently death. Important pathogens are Schistosoma mansoni, Schistosoma haematobium and Schistosoma japonicum. Affected are the local population, tourists, people working for humanitarian aid organizations and military personnel. In the case of infections of humans, the infectious cercariae, which are present in the water of open bodies of water penetrate through the skin into the body.

Likewise problematic, in countries with a moderate climate, is the infection of humans by cercariae of various species of the genera Trichobilharzia and Ornithobilharzia, which can drill into the skin, causing dermatitis. Such infections occur during leisure activities on inland waters or sea coasts and during fishing, working on ponds or watering fields. In general, in many situations of daily life, contact of the skin with possibly contaminated/infected water is unavoidable.

Protection against penetration of the pathogens is, however, possible by pretreating the skin according to the invention with anthelmintic substances.

In the past, some compounds have already been tested for their suitabality for preventing infections with such parasites. However, the substances hitherto described for the purposes according to the invention are toxic if they get into the body either through the skin or orally:

Thus, for example, hexachlorophene has a lethal effect on cercariae of Schistosoma mansoni (Fripp, P. J. and Armstrong, F. I., The efficacy of hexachlorophene skin cleanser as a cercariae repellent. South African Med. J. 47: 1973, 526-527). Because of health risks, in particular liver damage, hexachlorophene cannot be used on the skin of humans. It is toxic on contact with the skin and when swallowed, may possibly cause deformities and is possibly carcinogenic [Commission of the European Community, Directive 93/72/EEC of Sep. 1, 1993, Annex Vol. I and II (EU Directive on Dangerous Substances) with amendments to 1999, Official Journal EUL258A, Volume 36, 16 October 1993, Amendments to 1997].

Niclosamide acts against the penetration of cercariae [Bruce, J. I. et al. (1992) Efficacy of niclosamide as a potential topical antipenetrant (TAP) against cercariae of Schistosoma mansoni in monkeys. Mem. Inst. Oswaldo Cruz 87:28, 1-289.] but is toxicologically objectionable since it may possibly cause inheritable genetic damage (Registry of Toxic Effects of Chemical Substances, National Institute of Occupational Safety and Health). Use on the skin in cases where the user is exposed to water has to be ruled out owing to the risk it poses to the environment, since niclosamide constitutes a water hazard [Federal Office for the Environment (Ed.), Catalogue of substances hazardous to water. LTwS No. 12 May 1996 with current amendments, Berlin 1996]. Accordingly, the compound has hitherto been used commercially in humans against cercariae.

N,N-Diethyl-m-toluamide (DEET) acts against cercariae of Schistosoma mansoni [Salafsky, B. et al. Evaluation of N,N-diethyl-m-toluamide (DEET) as a topical agent for preventing skin penetration by cercariae of Schistosoma mansoni. Am. J. Trop. Med. Hyg. 58: 1998, 828-834). However, DEET has some unfavourable properties.

The effect of the anthelmintics hitherto described against infectious stages of platyhelminths has hitherto only been tested on cercariae of the species Schistosoma mansoni, i.e. an efficacy of these agents against other worm species had hitherto not been demonstrated.

Surprisingly, it has now been found that the compositions according to the invention are suitable for protecting humans and animals effectively against infections by platyhelminths, in particular Schistosoma haematobium, Schistosoma japonicum, Trichobilharzia spp. and Ornithobilharzia spp., but also Echinostoma spp. and others.

Accordingly, the invention relates to

1. Compositions for deterring helmintic parasites, characterized in that they comprise at least one compound of the formula (I)

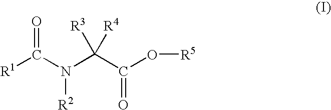

in which

R¹ represents hydrogen or an optionally substituted alkyl, alkenyl or alkinyl radical,

R² and R⁵ are identical or different and represent optionally substituted alkyl, alkenyl or alkinyl radicals and

R³ and R⁵ represent hydrogen, optionally substituted alkyl, aralkyl or aryl or, together with the atoms to which they are attached, form an optionally substituted monocyclic ring.

2. Compositions for deterring helmintic parasites according to item 1, characterized in that in the formula (I)

R¹ represents hydrogen, (C₁-C₁₀)-alkyl, (C₂-C₁₀)-alkinyl,

R² and R⁵ are identical or different and represent (C₁-C₁₀)-alkyl, (C₂-C₁₀)-alkenyl or (C₂-C₁₀)-alkinyl and

R³ and R⁴ represent hydrogen, (C₁-C₁₀)-alkyl, aryl or aralkyl, each of which is optionally mono- to trisubstituted in the aryl moiety by identical or different substituents and each of which has 6 to 10 carbon atoms in the aryl moiety and optionally 1 to 4 carbon atoms in the alkyl moiety, suitable aryl substituents being in each case: halogen, in each case straight-chain or branched (C₁-C₄)-alkyl, (C₁-C₄)-halogenoalkyl, (C₁-C₄)-halogenoalkoxy and (C₁-C₄)-halogenoalkylthio, or together with the atom to which they are attached represent cycloalkyl having 3 to 7 carbon atoms.

3. Compositions for deterring helmintic parasites according to item 1, characterized in that in the formula (I)

R¹ represents hydrogen, (C₁-C₅)-alkyl, (C₂-C₅)-alkenyl or (C₂-C₅)-alkinyl,

R² represents (C₁-C₅)-alkyl, (C₂-C₅)-alkenyl or (C₂-C₅)-alkinyl,

R³ and R⁴ represent hydrogen, (C₁-C₅)-alkyl, represent phenyl or phenylalkyl having optionally 1 or 2 carbon atoms in the alkyl moiety, each of which radicals is optionally mono- to trisubstituted in the phenyl moiety by identical or different substituents, suitable phenyl substituents being in each case: fluorine, chlorine, bromine, (C₁-C₂)-alkyl, (C₁-C₂)-halogenoalkyl, (C₁-C₂)-halogenoalkoxy, (C₁-C₄)-halogeno-alkylthio, or together with the atom to which they are attached represent cycloalkyl having 3 to 6 carbon atoms,

R⁵ represents (C₁-C₁₀)-alkyl, (C₂-C₅)-alkenyl or (C₂-C₅)-alkinyl.

4. Compositions for deterring helmintic parasites according to item 1, characterized in that in the formula (I)

R¹ represents hydrogen, (C₁-C₅)-alkyl, (C₂-C₅)-alkenyl or (C₂-C₅)-alkinyl,

R² represents (C₁-C₅)-alkyl, (C₂-C₅)-alkenyl or (C₂-C₅)-alkinyl,

R³ and R⁴ represent hydrogen, (C₁-C₅)-alkyl, represent phenyl, benzyl, phenethyl, each of which is optionally mono- to trisubstituted in the phenyl moiety by identical or different substituents, suitable substituents being in each case: fluorine, chlorine, bromine, methyl, ethyl, trifluoromethyl, trifluoromethoxy, trifluoromethylthio, or together with the atom to which they are attached represent cyclopropyl, cyclopentyl, cyclohexyl, and

R⁵ represents (C₁-C₁₀)-alkyl, (C₂-C₅)-alkenyl or (C₂-C₅)-alkinyl.

5. Compositions for deterring helmintic parasites according to item 1, characterized in that in the formula (I)

R¹ represents hydrogen, (C₁-C₅)-alkyl or (C₂-C₅)-alkenyl,

R² represents (C₁-C₅)-alkyl or (C₂-C₅)-alkenyl,

R³ and R⁴ represent hydrogen, (C₁-C₅)-alkyl, or together with the atom to which they are attached represent cyclopropyl, and

R⁵ represents (C₁-C₅)-alkyl or (C₂-C₅)-alkenyl.

6. A method for deterring helmintic parasites, characterized in that compounds of the general formula (I) are used.

7. The use of compounds of the general formula (I) for deterring helmintic parasites.

8. A process for preparing compositions for deterring helminthic parasites, characterized in that compounds of the general formula (I) are mixed with extenders and/or surfactants.

Particularly suitable for use in the compositions according to the invention is the compound of the formula

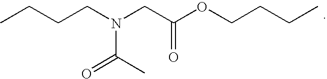

The compounds of the formula (I) and their preparation are known from German Offenlegungsschrift 38 20 528.

The active compounds contained in the compositions according to the invention have already been used specifically as repellents on the skin, against insects and ticks.

A substantial advantage of using the compounds according to the invention is their high compatibility with the skin, plants and the environment and the generally low toxicity of these compounds.

When staying outdoors, it is furthermore desirable to be protected against mosquitoes which, on the one hand, are considered a nuisance and, on the other hand, specifically in tropical regions, may transfer diseases such as malaria, various viruses, filaria and parasites by means of their sting. The compositions according to the invention now allow simultaneous prevention of platyhelminth infection and protection against mosquitoes, with only one composition. Thus, the necessity for using simultaneously two different, possibly incompatible compositions on the skin is avoided.

In addition to the active compounds, the compositions according to the invention may also comprise all of the customary auxiliaries and additives used in formulations for topical application.

The active compounds are administered, either directly or in the form of suitable preparations, dermally or with the aid of shaped articles containing the active compound, such as, for example, strips, plates, tapes, collars, ear tags, limb bands or marking devices.

Dermal administration is effected, for example, in the form of bathing, dipping, spraying, pouring-on or spotting-on, washing, shampooing, or powdering.

Suitable preparations include:

solutions or concentrates for administration after dilution, solutions for use on the skin, pour-on formulations, gels;

emulsions and suspensions for dermal administration and also semisolid preparations;

formulations in which the active compound is incorporated in an ointment base or in an oil-in-water or water-in-oil emulsion base;

solid preparations, such as powders, shaped articles containing the active compound.

Solutions for use on the skin are applied drop by drop, brushed on, rubbed in, splashed on or sprayed on, or applied by dipping, bathing or washing.

The solutions are prepared by dissolving the active compound in a suitable solvent and adding, if appropriate, additives such as solubilizers, acids, bases, buffer salts, antioxidants, preservatives.

Suitable solvents include: physiologically acceptable solvents, such as water, alcohols, such as ethanol, butanol, benzyl alcohol, glycerol, hydrocarbons, propylene glycol, polyethylene glycols and N-methyl-pyrrolidone, and their mixtures.

If appropriate, the active compounds can also be dissolved in physiologically acceptable vegetable or synthetic oils.

Suitable solubilizers include: solvents which facilitate the dissolution of the active compound in the main solvent or which prevent precipitation of the active compound. Examples of solubilizers are polyvinylpyrrolidone, polyethoxylated castor oil and polyethoxylated sorbitan esters.

Suitable preservatives are: benzyl alcohol, trichlorobutanol, p-hydroxybenzoic esters or n-butanol.

It may be advantageous to add thickeners when preparing the solutions. Suitable thickeners are: inorganic thickeners, such as bentonites, colloidal silica, aluminium monostearate, or organic thickeners, such as cellulose derivatives, polyvinyl alcohols and their copolymers, acrylates and methacrylates.

Gels which are applied to the skin or smoothed on are prepared by adding such an amount of thickener to solutions which have been prepared as described above that a clear composition is formed which has an ointment-like consistency. The thickeners used are the thickeners indicated further above.

Pour-on and spot-on formulations are poured or splashed onto limited areas of the skin, the active compound distributing itself over the surface of the body.

Pour-on and spot-on formulations are prepared by dissolving, suspending or emulsifying the active compound in suitable skin-compatible solvents or solvent mixtures. If appropriate, other auxiliaries, such as colorants, antioxidants, photostabilizers or tackifiers are added.

Suitable solvents include: water, alkanols, glycols, polyethylene glycols, polypropylene glycols, glycerol, aromatic alcohols, such as benzyl alcohol, phenylethanol, phenoxyethanol, esters, such as ethyl acetate, butyl acetate, benzyl benzoate, ethers, such as alkylene glycol alkyl ethers, such as dipropylene glycol monomethyl ether, diethylene glycol monobutyl ether, ketones, such as acetone, methyl ethyl ketone, aromatic and/or aliphatic hydrocarbons, vegetable or synthetic oils, DMF, dimethylacetamide, N-methylpyrrolidone, 2-dimethyl-4-oxy-methylene-1,3-dioxolane.

Colorants are all colorants which can be dissolved or suspended and which are approved for use in animals.

Auxiliaries include spreading oils, such as isopropyl myristate, dipropylene glycol pelargonate, silicone oils, fatty acid esters, triglycerides or fatty alcohols.

Antioxidants are sulphites or metabisulphites, such as potassium metabisulphite, ascorbic acid, butylhydroxytoluene, butylhydroxyanisole, tocopherol.

Examples of photostabilizers are substances from the class of the benzophenones and novantisolic acid.

Tackifiers are, for example, cellulose derivatives, starch derivatives, polyacrylates, natural polymers such as alginates, gelatin.

Emulsions are either of the water-in-oil or the oil-in-water type.

They are prepared by dissolving the active compound either in the hydrophobic or in the hydrophilic phase and by homogenizing this phase with the solvent of the other phase, with the aid of suitable emulsifiers and, if appropriate, other auxiliaries, such as colorants, bioabsorption promoters, preservatives, antioxidants, photostabilizers, and viscosity-increasing substances.

Suitable hydrophobic phases (oils) include: paraffin oils, silicone oils, natural vegetable oils, such as sesame seed oil, almond oil, castor oil, synthetic triglycerides, such as caprylic/capric acid biglyceride, a triglyceride mixture with vegetable fatty acids of chain length C₈-₁₂ or other specifically selected natural fatty acids, mixtures of partial glycerides of saturated or unsaturated fatty acids which may also contain hydroxyl groups, mono- and diglycerides of the C8/C₁₀-fatty acids.

Fatty acid esters, such as ethyl stearate, di-n-butyryl adipate, hexyl laurate, dipropylene glycol pelargonate, esters of a branched fatty acid having a medium chain length with saturated fatty alcohols of chain length C₁₆-C18, isopropyl myristate, isopropyl palmitate, caprylic/capric esters of saturated fatty alcohols of chain length C₁₂-C₁₈. isopropyl stearate, oleyl oleate, decyl oleate, ethyl oleate, ethyl lactate, waxy fatty acid esters such as dibutyl phthalate, diisopropyl adipate, ester mixtures related to the latter, amongst others fatty alcohols, such as isotridecyl alcohol, 2-otyldodecanol, cetylstearyl alcohol or oleyl alcohol.

Fatty acids, such as, for example oleic acid and its mixtures.

Suitable hydrophilic phases include:

water, alcohols, such as, for example, propylene glycol, glycerol, sorbitol and their mixtures.

Suitable emulsifiers include: nonionic surfactants, for example polyethoxylated castor oil, polyethoxylated sorbitan monooleate, sorbitan monostearate, glycerol mono-stearate, polyethoxy stearate, alkylphenol polyglycol ethers;

ampholytic surfactants, such as disodium N-lauryl-B-iminodipropionate or lecithin;

anionic surfactants, such as sodium lauryl sulphate, fatty alcohol ether sulphates, and the monoethanolamine salt of mono/dialkylpolyglycol ether orthophosphoric ester;

cationic surfactants, such as cetyltrimethylammonium chloride.

Other suitable auxiliaries include: substances which increase the viscosity and stabilize the emulsion, such as carboxymethylcellulose, methylcellulose and other cellulose and starch derivatives, polyacrylates, alginates, gelatin, gum arabic, polyvinylpyrrolidone, polyvinyl alcohol, methylvinyl ether/maleic anhydride copolymers, polyethylene glycols, waxes, colloidal silica, or mixtures of the listed substances.

Suspensions are prepared by suspending the active compound in a liquid excipient, if appropriate with the addition of other auxiliaries, such as wetting agents, colorants, bioabsorption promoters, preservatives, antioxidants and photostabilizers.

Suitable liquid excipients include all homogeneous solvents and solvent mixtures.

Suitable wetting agents (dispersants) include the surfactants indicated further above.

Other suitable auxiliaries include those indicated further above.

Semi-solid preparations for dermal administration differ from the suspensions and emulsions described above only in that they have a higher viscosity.

To prepare solid preparations, the active compound is mixed with suitable excipients, if appropriate with the addition of auxiliaries, and the mixture is formulated as desired.

Suitable excipients include all physiologically acceptable solid inert substances.

Suitable for this purpose are inorganic and organic substances. Inorganic substances are, for example, sodium chloride, carbonates, such as calcium carbonate, hydrogen carbonates, aluminium oxides, silicas, clays, precipitated or colloidal silica, and phosphates.

Auxiliaries are preservatives, antioxidants and colorants which have already been mentioned further above.

Other suitable auxiliaries are lubricants and glidants, such as, for example, magnesium stearate, stearic acid, talc, bentonites.

It is furthermore desirable for such a protective agent to have a sufficient protective action even after prolonged contact with water, for example when swimming, washing clothes or fishing. To this end, the compositions according to the invention may additionally comprise water-repelling or water-proof substances.

Suitable water-proof substances are already being used in sun protection compositions which are to protect the user against the UV radiation from the sun (for example U.S. Pat. No. 5,518,712 and U.S. Pat. No. 4,810,489). Here, it is intended to maintain sun protection even after the user has been swimming, is sweating heavily, etc. Sun protection compositions which comprise such water-proof or water-repelling substances and insect repellents are already known (U.S. Pat. No. 5,716,602). However, compositions which comprise anthelmintics have hitherto not been described.

Accordingly, water-proof substances may also be present in the composition according to the invention. These may be substances which are soluble in fat and insoluble in water, and compounds which improve adherence of the composition to the skin.

Skin protection products may comprise, as water-proof components, for example from 1 to 50% by weight of a polymer such as polyvinylpyrrolidones, polyacrylates, silicones, etc.

The compositions for topical application can be formulated as sprays, solutions, creams, ointments or layer- or film-forming compositions, according to the known processes for manufacturing cosmetics (Schrader, K. (1979) Grundlagen und Rezepturen der Kosmetika [Principles of and recipes for cosmetics], Dr. Alfred Hüthig Verlag, Heidelberg).

For use, the formulations according to the invention are applied evenly and without any gaps onto the skin, in amounts appropriate for the user.

The compositions according to the invention are of course also suitable for use on animals to prevent infection of the animals with parasites of these genera. The compositions can be used for pets, such as, for example, dogs and cats, and for economically useful animals, for example cattle, sheep, etc.

When using the compositions according to the invention, in general from 0.03 to 1 mg, preferably from 0.03 to 0.1 mg and particularly preferably from 0.04 to 0.06 mg of the active compound are applied per cm² of skin. This results in prophylactic protection against skin-penetrating worms and juvenile stages thereof. If the user stays in the water for a longer time, the active compound has to be applied repeatedly.

The examples below illustrate the compositions according to the invention, but without limiting them.

### BIOLOGICAL EXAMPLE

Activity Against Schistosoma mansoni cercariae

[500 μl/l Final Concentration of the Active Compounds]

Snails (Biomphalaria glabrata) were infected by incubating each of them with 8 miracidia in 10 ml of water overnight. About 6 to 9 weeks after the infection, cercariae were obtained by irradiating the snails, which had been kept in darkness, with light, followed by collection of the swarming cercariae within 2 hours.

Such an amount of cercariae-containing water (1 or 2 ml, see below) was added to the test batches that each batch contained about 100 to 150 cercariae.

5 μl of active compound were mixed thoroughly with 25 μl of PEG300. 9 ml of aquarium water was then added, and the batch was shaken vigorously. After (delayed) addition of 1 ml of cercariae suspension, survival of the cercariae was in each case observed immediately using a stereomagnifier. The activity of the active compounds was assessed using the following classification: 0=no effect during the entire test period of 120 minutes; I=weak effect (the cercariae have a strongly reduced mobility); 2=good effect (the cercariae are only slightly mobile and bent); 3=full effect (the cercariae are completely immobile).

In this test, the effect of the compound of the following formula

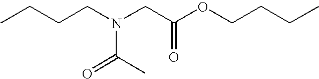

was rated 1.

1. Compositions for deterring helmintic parasites, characterized in that they comprise at least one compound of the formula (I) 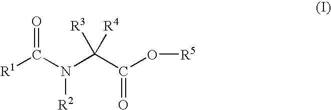
in which
R¹ represents hydrogen or an optionally substituted alkyl, alkenyl or alkinyl radical,
R² and R⁵ are identical or different and represent optionally substituted alkyl, alkenyl or alkinyl radicals and
R³ and R⁵ represent hydrogen, optionally substituted alkyl, aralkyl or aryl or, together with the atoms to which they are attached, form an optionally substituted monocyclic ring.

2. Compositions for deterring helmintic parasites according to claim 1, characterized in that in the formula (I)
R¹ represents hydrogen, (C₁-C₁₀)-alkyl, (C₂-C₁₀)-alkinyl,
R² and R⁵ are identical or different and represent (C₁-C₁₀)-alkyl, (C₂-C₁₀)-alkenyl or (C₂-C₁₀)-alkinyl and
R³ and R⁴ represent hydrogen, (C₁-C₁₀)-alkyl, aryl or aralkyl, each of which is optionally mono- to trisubstituted in the aryl moiety by identical or different substituents and each of which has 6 to 10 carbon atoms in the aryl moiety and optionally 1 to 4 carbon atoms in the alkyl moiety, suitable aryl substituents being in each case: halogen, in each case straight-chain or branched (C₁-C₄)-alkyl, (C₁-C₄)-halogenoalkyl, (C₁-C₄)-halogenoalkoxy and (C₁-C₄)-halogenoalkylthio, or together with the atom to which they are attached represent cycloalkyl having 3 to 7 carbon atoms.

3. Compositions for deterring helmintic parasites according to claim 1, characterized in that in the formula (I)
R¹ represents hydrogen, (C₁-C₅)-alkyl, (C₂-C₅)-alkenyl or (C₂-C₅)-alkinyl,
R² represents (C₁-C₅)-alkyl, (C₂-C₅)-alkenyl or (C₂-C₅)-alkinyl,
R³ and R⁴ represent hydrogen, (C₁-C₅)-alkyl, represent phenyl or phenylalkyl, each of which is optionally mono- to trisubstituted in the phenyl moiety by identical or different substituents and each of which has optionally 1 or 2 carbon atoms in the alkyl moiety, suitable phenyl substituents being in each case: fluorine, chlorine, bromine, (C₁-C₂)-alkyl, (C₁-C₂)-halogenoalkyl, (C₁-C₂)-halogenoalkoxy, (C₁-C₄)-halogenoalkylthio, or together with the atom to which they are attached represent cycloalkyl having 3 to 6 carbon atoms,
R⁵ represents (C₁-C₁₀)-alkyl, (C₂-C₅)-alkenyl or (C₂-C₅)-alkinyl.

4. Compositions for deterring helmintic parasites according to claim 1, characterized in that in the formula (I)
R¹ represents hydrogen, (C₁-C₅)-alkyl, (C₂-C₅)-alkenyl or (C₂-C₅)-alkinyl,
R² represents (C₁-C₅)-alkyl, (C₂-C₅)-alkenyl or (C₂-C₅)-alkinyl,
R³ and R⁴ represent hydrogen, (C₁-C₅)-alkyl, represent phenyl, benzyl, phenethyl, each of which is optionally mono- to trisubstituted in the phenyl moiety by identical or different substituents, suitable substituents being in each case: fluorine, chlorine, bromine, methyl, ethyl, trifluoromethyl, trifluoromethoxy, trifluoromethylthio, or together with the atom to which they are attached represent cyclopropyl, cyclopentyl, cyclohexyl, and
R⁵ represents (C₁-C₁₀)-alkyl, (C₂-C₅)-alkenyl or (C₂-C₅)-alkinyl.

5. Compositions for deterring helmintic parasites according to claim 1, characterized in that in the formula (I)
R¹ represents hydrogen, (C₁-C₅)-alkyl or (C₂-C₅)-alkenyl,
R² represents (C₁-C₅)-alkyl or (C₂-C₅)-alkenyl,
R³ and R⁴ represent hydrogen, (C₁-C₅)-alkyl, or together with the atom to which they are attached represent cyclopropyl, and
R⁵ represents (C₁-C₅)-alkyl or (C₂-C₅)-alkenyl.

6. A method for deterring helmintic parasites, characterized in that compounds of the general formula (I) are used.

7. Use of compounds of the general formula (I) for deterring helmintic parasites.

8. Process for preparing compositions for deterring helminthic parasites, characterized in that compounds of the general formula (I) are mixed with
